(19) United States
(12) Patent Application Publication
(10) Pub. No.: US 2015/0126975 A1
(43) Pub. Date: May 7, 2015

(54) GRAVITY URINARY DRAINAGE SYSTEM

(71) Applicant: Sienna Colorado Solutions, LLC, Colorado Springs, CO (US)

(72) Inventor: Phil Wuthier, Colorado Springs, CO (US)

(73) Assignee: SIENNA COLORADO SOLUTIONS, LLC, Colorado Springs, CO (US)

(21) Appl. No.: 14/532,728

(22) Filed: Nov. 4, 2014

Related U.S. Application Data
(60) Provisional application No. 61/900,169, filed on Nov. 5, 2013.

(51) Int. Cl. A61F 5/44

(52) U.S. Cl. CPC A61F 5/4404
USPC 604/544

(57) ABSTRACT

An improved fluid drainage system is comprised of a transfer tubing that includes a secondary axial compartment for the movement of air. This secondary lumen of the transfer tubing includes a gas permeable material which will allow the transport of air, while repelling fluid. The secondary lumen transports air from any section of the transfer tubing to the collection bag, as a means of eliminating and equating pressurized air pockets that can result in flow retardation. The urinary drainage system of the present invention remains a closed system, but with an internal venting system to improve flow distally from the body.

### CROSS-REFERENCE TO RELATED APPLICATIONS

The present application relates to and claims the benefit of priority to U.S. Provisional Application No. 61/900,169, Gravity Urinary Drainage System, filed Nov. 5, 2013 which is hereby incorporated by reference in its entirety for all purposes as if fully set forth herein.

### BACKGROUND OF THE INVENTION

1. Field of the Invention

Embodiments of the present invention pertain to field of endeavor of medical drainage devices and more particularly to gravity driven urinary drainage devices and methodology.

2. Description of Relevant Art

The indwelling urinary drainage catheter, also commonly called the Foley catheter, is a well-accepted medical device frequently used in hospital and clinical settings. It is considered to be an essential part of a patient's medical care, but also been called an often-ignored and omnipresent device. The indwelling catheter is comprised of the Foley catheter (catheter tip), transfer tubing, and collection bag. In established standard practice, the catheter is aseptically inserted into the patient's bladder via the urethra, and secured to the patient's upper thigh. The transfer tubing and collection bag are positioned and secured in a dependent location, never to be elevated above the patient's bladder. Ideally, urine from the bladder freely drains by gravity flow through the system into the collection bag.

In practice the components utilized in urinary tract drainage devices—transfer tubing and drainage bag—are used more widely to drain body fluids from a patient in a gravity driven fashion. In actual medical practice these components may be identical or slightly modified from the configuration of the urinary drainage system.

In the medical plan of care, urinary drainage systems are ubiquitous. Between 16 and 25% of all hospitalized patients will have an indwelling catheter inserted sometime during their stay. Consequently, as many as 30 million catheters are inserted annually in United States hospitals and extended care settings. Worldwide usage is estimated at 96 million, with the expectation that numbers will increase in future years with an aging population.

The modern catheter was invented by Dr. Frederick Foley in 1927 to reduce bleeding following prostate transurethral resection surgery, and included a one piece inflating balloon to anchor the unit inside the bladder. In its early use, a catheter protruding from a patient's urethra was extended to an open tray to catch the effluent urine. With this open style drainage system it was later found that the incidence of urinary tract infection (UTI) in the patient occurred in 100% of cases within several days of insertion. The use of a closed drainage system was not widely used until the late 1960's. Since that time, there is established practice of only using a closed system configuration, namely, a Foley catheter connected directly to a transfer tube, which is connected directly to a closed (but vented) drainage bag. It is standard medical practice to avoid disconnecting the components while in use, so as to minimize any breaks in the closed configuration. This important practice stems from the historically known high infection incidence of an open system, as previously described above.

Despite the many known limitations of the current urinary catheter (detailed herein) and the advances in medical technology and medical understanding in recent years, the catheter physical configuration has remained larger unimproved since 1937. The photographic plate showing Dr. Foley's innovative catheter as presented in the aforementioned 1937-journal article, might be mistaken for a present-day catheter (less the balloon filling check valve made possible by modern plastics technology). The majority of current efforts at improving the urinary drainage system have focused on changing and improving the material coating of the inserted catheter so as to aid in the catheter's initial application and deter exterior sources of infection. There seems common agreement in urological literature with one group of authors' assertion that it is difficult to understand why we are still unable to perform the relatively simple task of draining urine from the bladder without producing infection and a range of associated complications.

Catheter associated urinary tract infections (CA-UTI) are a well-documented problem with urinary drainage systems. Urinary tract infections are the most prevalent of all nosocomial infections with 95% of those infections due to urinary catheter placement and use. More than a million U.S. patients annually suffer the burden of increase morbidity and increased treatment costs due to these nosocomial CA-UTI. Furthermore, CA-UTI are the second most common cause of nosocomial bloodstream infections. However, only 4% of all CA-UTI result in bacteremia. Although CA-UTI are frequently asymptomatic—at least until the catheter is removed—they can represent a significant source of antibiotic-resistant yeast and bacteria within the body. It is not fully understood why symptoms of infection present in some individuals and not in others, but it is clear from clinical studies that the presence of the catheter makes the individual much more susceptible to progressive infection. Whereas, a non-catheterized individual who experiences a breach of bacteria into the bladder (bacteriuria) seldom develops accelerated bacterial growth, the catheterized individual is likely to have a rapid progression of infection to greater than 10⁵ cfu/ml. This amount is considered high-level bacteriuria. Presumably, this vulnerability is due to the fact that the catheter circumvents the bladder and ureter's natural ability to effectively flush the bacteria from the bladder. Asymptomatic bacteriuria puts a patient at risk for a host of more serious complications, including pyelonephritis, bacteremia, septicemia, catheter encrustation and obstruction, stone formation, urethral strictures, urethritis, periurethral abscess, prostatitis and the acquisition of Candida and multidrug-resistant bacteria. The presence of a reservoir of antibiotic resistant organisms in the bladder may be the greatest, although silent threat of bacteriuria.

A number of terms are used in the technical description of the presence of bacteria in the bladder. Although these terms are often used interchangeable within published literature, careful definition is important. Several related definitions exist, but CA-UTI is commonly defined for a catheterized patient as a positive urine culture growing more than 10³ cfu/ml of more than one organism, along with the symptoms of UTI (elevated temperature, altered mental status, urinary frequency or urgency, suprapubic tenderness, or dysuria) with no other identifiable infection source. Asymptomatic catheter associated bacteriuria (CA-ASB) is defined in a patient with a positive urine culture of greater than 10⁵ cfu with none of the symptoms of a UTI present. These definitions form part of the “2009 International Clinical Practice Guidelines” for the Infectious Disease Society of America.

Contemporary studies affirm that the pathogenesis of catheterized bacteriuria remains poorly understood by the Microbiology community. What appears to be accepted is as follows: CA-UTI and CA-ASB can occur either due to extraluminal or intraluminal contamination. Extraluminal contaminations typically occurs either soon after catheter insertion (presumably due to non-aseptic insertion in which bacteria residing near the meatal orifice are transported with the catheter to the bladder) or after a significant number of days (presumably due to the development of a biofilm on the outside of the catheter growing up towards the bladder). The warm, moist conditions that exist between that direct contact of the urethra and the catheter provide an optimal environment for biofilm growth. Intraluminal contaminations appear between these infection times of the extraluminal infections, and are presumed to occur due to reflux of contaminated urine from the collection bag up towards the bladder. It is significant to note that the intraluminal contamination was determined to be a non-biofilm driven infection route. Furthermore, it is significant to note that bacteria from the intraluminal route occur more rapidly than the seemingly ideal growth conditions of the extraluminal route. Extraluminal contamination has been identified to account for 66% of CA-UTI, and typically result from the patient's own perineal flora. Intraluminal contamination accounts for the remaining 34% of contamination cases, and were distinguished from the extraluminal cases because of different species of contaminating bacteria. Both routes are deemed important in efforts to reduce CA-UTI.

The method of intraluminal contamination has yet to be identified or confirmed. The presence of primarily exogenous bacteria in infections identified to be intraluminal in origin, are consistent with contamination from the hands of health care workers. Such infections have been presumed to be result from retrograde urine flow back into the bladder when the catheter bag is moved or adjusted, but there has been no definitive explanation. While the exact intraluminal mechanism remains undefined in professional literature, both animal and human studies have demonstrated that bacteria that enter the drainage bag are soon found in the bladder. Efforts and claims made herein will primarily address the reduction of intraluminal contamination.

Complete bladder emptying during urination is widely known as one of the body's main defenses against bacteriuria and urinary retention and is a common complication for elderly and hospital bound patients. There are a wide range of reasons for urine retention in the bladder, including inflammatory, pharmacologic, neurological, and infectious processes. Trauma and post-operative complications also can cause retention. Urinary catheter related retention issues include mineral incrustation in long term usage, and mechanical blockages due to kinks and air-locks.

Research studies evaluating the possible adverse effects of urinary retention on patient outcomes continue to build empirical knowledge on retention. Results of such studies are not always conclusive or in agreement as to the relationship of urinary retention and bladder infection in affected adult individuals. However many studies have determined a clear correlation in specific populations. The association of retention and UTI in pediatric patients is deemed irrefutable and established. A recent study of adult males concluded that a post void residual (urinary retention) of 180 ml or more predisposed otherwise asymptomatic men to a high risk for bacteriuria. A similar study confirmed the positive correlation in adult males between retained urine and UTI, but was not able to establish a critical volume amount. Similarly, a study of women with identified UTI found that they also had post void residual issues. The women with a UTI had a residual twice the average of the non-UTI patients. A study of elderly, hip fracture patients found 38% to have urinary retention. In a descriptive nursing chart audit study, the only statistically significant indicator of UTI was catheter blockage and low urine output. Thus, there is growing evidence to support that blocked urinary flow (urinary retention) is associated with bacteria in the bladder.

There has been a recent increased focus on providing urinary catheter patients in hospital and clinical setting with rigorous and vigilant nursing care in order to avoid CA-UTI. This has been driven by a greater awareness of the predisposition of catheterized patients to infection, as well as restrictions on the cost reimbursement for such acquired infections. One aspect of this focus is the pursuit of eliminating dependent loops in the urinary catheter transfer tubing. A dependent loop is defined as any point where the transfer tubing dips below the level of its entry point into the catheter bag.

Hospital nursing procedural teaching is well established for eliminating these catheter tubing dips with a view of providing continuous downward flow progression from the secured catheter (typically on the patients anterior thigh) to the location of the collection bag (typically on the bed frame). Such principles are widespread, although presented without evidenced based explanation. The recommendation is often accompanied by the warning that the catheter bag must always remain below the level of the patient's bladder, whether in simple use or in the transfer of patients. Both efforts are asserted as a means of ensuring only ante-grade, unrestricted flow from the patient's bladder to the collection bag. In spite of both manufacturer and hospital instruction the use of dependent loops remains prevalent. Indeed, one source reported dependent loops in 85% of observed patients with catheters.

In an extensive and unique medical study of 850 newly catheterized patients, risk factor and nursing interventions were evaluated as to their effect on CA-UTI. The study concluded: “The only catheter-care violation predictive of an increased risk for CAUTI was the drainage tube sagging below the level of the collection bag.” This is a very significant finding from a large scale study. This study points to the critical issue of dependent loops in catheterized patients, without formally identifying the means of transmission of the infecting bacteria.

With the existence of a dependent loop in the transfer tubing of the catheter, it is a frequent clinical observation that after urine has filled the lowest section of the tubing, a pressure differential will exist between the two legs of the dependent loop. This is evidenced by differing water leg levels. In the typical arrangement, the water leg proximal to the patient is at a higher pressure (displayed by a smaller height of liquid column from the lowest point of the tubing). One source cites this situation to occur in 65% of observed cases. It is clear from observation, that as the bladder contracts and increases the proximal air tubing pressure, the pressure will build until either: 1) fluid rising in the distal leg crests into the collection bag (resulting in a fixed applied pressure situation), 2) the proximal leg decreases to the lowest point in the tubing whereby pressurized air in bubbles crosses to the distal leg, temporarily relieving the built-up pressure (usually only occurring when the tubing has been completely emptied and urine is beginning to fill the most inferior portion of the tubing), or 3) the bladder applies (and holds) its maximum available pressure, resulting in a stable pressure differential across the dependent loop. In the latter case, the urinary drainage system is air-locked, and no further urine flow will occur.

A more infrequent clinical observation is the presence of a reverse pressurization to that described above. In this scenario, the proximal liquid leg height exceeds the height of the distal leg indicating a lower pressure in the section of air between the patient and the dependent loop. This condition existed in 33% of observed case, while only 2% showed no pressure build-up. This situation exists where the catheter tip openings located within the bladder have become temporarily blocked by tissue. One source indicates that “negative pressure in the catheter can suck the bladder mucosa into the eye-holes of the catheter causing hemorrhagic pseudopolyps”. Thus a negative gauge pressure can exist between the patient and the dependent loop, and the consequences of its existence may prove unbeneficial to the patient.

The existence of dependent loops in the catheter transfer tubing are very common, and the pressure differentials from the patient across the loop may be either positive or negative in value. Both of these cases result in inhibited urine flow from the bladder tying the existence of the dependent loop to the incidence of bacteriuria.

Inadequate or delayed drainage of the bladder with a urinary catheter also results in patient discomfort. It has been observed by the inventor on numerous occasions, post-operative patients under the effects of anesthesia or with sensory decreasing maladies, feel the need to urinate with an air-locked urinary catheter in place. The nature of an air-locked catheter will be discussed further in the document, but such a situation where the flow of urine from the bladder is inhibited, causes the patient the discomfort of a full bladder with no means to relieve the discomfort. The urge to urinate occurs at a bladder volume of approximately 200 ml. Furthermore, a catheter attached to the patients leg and the collection bag hooked to the patient's bed acts as a one-point restraint limiting the patient's free movement. Nursing staff are required to frequently remind such sedated or debilitated patients of the presence of the catheter in order to avoid patient falls, as such patients have a tendency to try and get out of bed to urinate. Air-locked urinary drainage systems create patient discomfort and increase the risk of patient falls.

One area of current medical research is the field of transmission of bacterial contamination through the aerosol vector. In a medical setting it has been demonstrated that bacterial transmission is possible via aerosol vapors generated by a standard toilet flush, a simple hinged door opening or closing, and in water spray or shower head operation. These seemingly innocuous avenues of infection transmission all contain the existence of an aerosol vapor and a pressure as the driving force for dissemination. The aforementioned pressurization of the catheter transfer tubing, and the cycle of pressurization/depressurization that will occur with movement of the bag, patient movement, existence and resolution of air-lock, provide the driving force which may make transmission of aerosolized bacteria possible in the prior art.

A description of the nature of the hydraulics of flow through small diameter tubing is needed in order to understanding on the limitation of the existing drainage system. In the current configuration of the urinary drainage system, the flexible tubing utilized between the Foley catheter and collection bag is comprised of standard modern flexible plastic of small diameter. For tubing or piping in which air is entrapped within the liquid flow, the dimensionless parameter called the Froude number (FR) is utilized by engineers to determine the liquid flow velocity (also called “flushing velocity”) necessary to expel the air. The FR is calculated as the resultant of the liquid flow velocity divided by the square root of the product of the pipe internal diameter and gravitational constant. It thus represents a ratio of inertia and gravitational forces. A FR value greater than 0.35 is required to purge air from a vertical line. while a value greater than 1.0 is needed to purge air from a horizontal line.

Routine engineering calculations using the average volume of urine voided by an individual over a standard period of time, allow for approximate calculations of flow velocity. Numerous articles detail typical urine flow rates. Using these values and standard catheter tip inside diameters and transfer tubing inside diameters allows for the calculation of typical FR numbers that result during urination. See TABLE 1 for results. Such calculations show that typical urine flow rates generated by the bladder result in FR values sufficient to drive air from the catheter tip, but insufficient to drive air from the transfer tube. These calculations are consistent with clinical observations, which show the transfer tube routinely a mixture of two-phase flow (urine and air), and the junction between the transfer tube and catheter tip (at the location of the transition fitting) to be the transition to solid urine flow. When the patient's bladder is at rest and not forcing urine through the system, this internal diameter change is the boundary for which air does not typical proceed upstream. The significance of this point is discussed further in subsequent discussion.

TABLE 1
Urine Froude Number Calculations

| | | | TUBE | | |
|---|---|---|---|---|---|
| | | | DIMENSIONS | | |
| URINE FLOW | | | Inside | FROUDE CALCULATIONS | |
| Volume | Time | Flow | Diameter | Velocity | Froude Number |
| (ml) | (sec) | (ml/s) | (mm) | (m/s) | (—) |

CATHETER TIP:

| | | | | | |
|---|---|---|---|---|---|
| 200 | 10 | 27.69 | 2 | 0.637 | 4.55 |
| 200 | 15 | 18.46 | 2 | 0.424 | 3.03 |
| 200 | 30 | 9.23 | 2 | 0.212 | 1.52 |

TRANSFER TUBING:

| | | | | | |
|---|---|---|---|---|---|
| 200 | 10 | 27.69 | 8 | 0.040 | 0.14 |
| 200 | 15 | 18.46 | 8 | 0.027 | 0.01 |
| 200 | 30 | 9.23 | 8 | 0.013 | 0.05 |

CRITICAL DIAMETERS:

| | | | | | |
|---|---|---|---|---|---|
| 200 | 10 | 27.69 | 5.58 | 0.082 | 0.35 |
| 200 | 30 | 9.23 | 3.60 | 0.066 | 0.35 |
| 200 | 10 | 27.69 | 3.67 | 0.190 | 1.00 |
| 200 | 30 | 9.23 | 2.36 | 0.152 | 1.00 |

Flow through tubes of small diameter, such as that for the catheter tip or transfer tubing, is dominated by surface tension effects. For such small diameter dynamics a parameter more useful than FR is the Eotvos number (EO). This dimensionless parameter represents a ratio of buoyancy forces to surface tension forces. It is calculated as the product of the tube internal diameter squared, the gravitational constant and the difference between the liquid and gas densities, divided by the liquid surface tension. EO has been identified as a key parameter in evaluating two-phase flow in small diameter tubes.

The overall effect of the surface tension in the small diameter transfer tubing is a flow regime commonly designated as “slug flow” or “intermittent flow”. In this regime the drainage of fluid in a particular direction is accompanied by the passage of air bubbles moving in the opposite direction. These bubbles are commonly referred to in literature at “Taylor Bubbles” or “Dumitrescu Bubbles,” and have been studies for many years for a variety of application ranging World War II submarine explosions to present-day cooling of micro-processing devices. In a gravity flow situation such as exists for the drainage of the standard Foley catheter transfer tubing, the motion of these bubbles controls the amount and velocity of the urine proceeding towards the collection bag.

Of relevance in this discussion, is the existence of a critical EO value below which an air bubble will not rise in a gravitational liquid field. It was experimentally observed as early as 1913, that there existed a minimum tube diameter size below which a Taylor bubble would not rise. More recent theoretical and empirical studies have further clarified the critical condition. One such study identifies a critical EO value of 3.37. Using this critical EO value and published values of the range of urine surface tension, one can easily calculate the critical internal tube diameter for which an air bubble will not rise within urine. See TABLE 2 for these results. Note from the table that the typical transfer tubing internal diameter is above the critical values, and the typical Foley catheter lumen diameter is below the values.

TABLE 2
Urine Eotvos Number Calculations

| URINE PROPERTIES | | | | | |
|---|---|---|---|---|---|
| Urine Specific Gravity | 1.005 | 1.01 | 1.015 | 1.02 | 1.025 |
| Urine Surface Tension | 67 | 64.25 | 61.5 | 58.75 | 56 |
| (dynes/cm) | | | | | |
| CATHETER | INTERNAL DIAMETER = 2 mm | | | | |
| Eotvos Number | 0.59 | 0.62 | 0.65 | 0.68 | 0.72 |
| TRANSFER TUBING | INTERNAL DIAMETER = 8 mm | | | | |
| Eotvos Number | 9.41 | 9.86 | 10.35 | 10.89 | 11.48 |
| CRITICAL DIAMETER | EOTVOS = 3.37 | | | | |
| Internal Diameter (mm) | 4.79 | 4.67 | 4.56 | 4.45 | 4.33 |

This finding highlights the fact that under gravitation influence alone, an air bubble will rise through the transfer tubing allowing for Taylor Bubble type drainage, but will not progress upstream of the Foley catheter transition junction. This is consistent with clinical observation of patients with urinary catheters, that there is a stable air-urine transition point near the location where the internal diameter decreases into the Foley catheter.

A very significant consequence of this physical phenomenon, is that in order to overcome this critical feature of small diameter surface tension controlled flow, pressure is required. Pressure from the bladder forces fluid through the smaller diameter into the transfer tubing for the desired ante-grade flow toward the collection bag. Similarly, with the prior art, any distally applied pressure to the stable air-urine boundary has the propensity to drive either air or liquid back into the bladder. However applied (whether by manipulation of the transfer tubing or drainage bag, or other means), potentially contaminated fluid or air can be propelled back towards the bladder when a distal pressure exists greater than that supplied by the bladder at the time in question (see also the previous discussion on aerosol bacterial contamination).

Accordingly, typical urination flow rates are not sufficient to drive the air out of the catheter transfer tubing, meaning that flow will be of a two-phase (urine and air) nature. The size of the transfer tubing is such that surface tension effects and gravity effect work together in controlling flow characteristics. Further, the diameter of the interior of the Foley catheter itself is clearly below the critical EO value, meaning that two-phase flow will not exist within the catheter tip without a driving pressure force. In other words, for retrograde flow of air or urine back into the bladder a distal pressure greater than that provided by the bladder itself, would be required. A need therefore exists to provide an environment for continual flow of urine from the bladder to the collection bag. Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### BRIEF SUMMARY OF THE INVENTION

The present invention is an improved medical drainage system for the drainage of a body fluid from the orifice of a patient. In the various embodiments described herein, this invention will be part of a system which can be inserted into the bladder or other body orifice for body fluid drainage. The most typical application involves the drainage of urine from the bladder via a catheter inserted into the urethral meatus and proceeding to the bladder. The components of the system addressed in this invention involve the transfer tubing and fitting, and the collection bag.

According to one embodiment of the present invention a system for enhanced drainage of bodily fluids includes a fluid extraction device operable to remove a fluid from a fluid source coupled to a fluid collection device by length of conduit. The conduit includes an interior channel that is separated along the length of the conduit by a selectively permeable barrier or membrane. This membrane or barrier defines a primary and secondary lumen within the conduit. Through the secondary lumen liquid vapor and gases freely travel enabling the pressure throughout the length of the conduit to remain constant facilitating fluid flow.

The selectively permeable barrier is, according to one embodiment of the present invention, permeable to a vapor state of a substance and impermeable to a liquid state of the same substance. The barrier thus maintains equal pressure throughout the conduit. While the present invention is described below in terms of a means to remove urine from a patient using a catheter as the source of the fluid one skilled in the relevant art will recognize that the concepts disclosed herein can be applied to any extraction or eradication of fluid from the body. In the embodiment in which the invention is employed to faciliate urine removal from a patient the secondary lumen within the conduit remains void of urine so as to equalize pressure while the primary lumen within the conduit conveys the urine to the collection bag. By doing so the selectively permeable barrier reduces aerosole transmission of bacteria within the conduit as well as eliminating vacuum driven adherence of the catheter tip to a bladder mucosa.

One or more embodiments of the present invention allows for more effective drainage of the bladder. Within the lumen of the transfer tubing a secondary lumen is utilized. In the various embodiments, this secondary lumen allows for the transport of air (only) from any location in the transfer tubing away from the patient to the collection bag. With the system in place for the drainage of fluid from a body orifice, the initiation of a pressure build-up in the tubing will result in quick and effective pressure dissipation (or prevention) through the secondary lumen into the collection bag. Accordingly, the present invention greatly improves drainage through the transfer tubing towards the collection bag. Undesirable retrograde flow is not enhanced by the proposed invention.

The features and advantages described in this disclosure and in the following detailed description are not all-inclusive. Many additional features and advantages will be apparent to one of ordinary skill in the relevant art in view of the drawings, specification, and claims hereof. Moreover, it should be noted that the language used in the specification has been principally selected for readability and instructional purposes and may not have been selected to delineate or circumscribe the inventive subject matter; reference to the claims is necessary to determine such inventive subject matter.

### BRIEF DESCRIPTION OF THE SEVERAL VIEWS OF THE DRAWING

The aforementioned and other features and objects of the present invention and the manner of attaining them will become more apparent, and the invention itself will be best understood, by reference to the following description of one or more embodiments taken in conjunction with the accompanying drawings, wherein:

FIG. 1 is a simplified view of the components of a urinary drainage system according to one embodiment of the present invention;

FIG. 2 is a cross sectional-view along LINE 1-1 of FIG. 1, of three possible embodiments of the improved design urinary drainage system according to the present invention;

FIG. 3 is a cross sectional-view along LINE 1-1 of FIG. 1 illustrating the conveyance of differing phases of a substance across a selectively permeable barrier within a conduit according to one embodiment of the present invention;

FIG. 4 shows a typical longitudinal view, according to one embodiment of the present invention, of transfer tubing showing typical system functionality of the improved design urinary drainage system;

FIG. 5 shows side cut-away view of a transitional fitting between the transfer tubing, according to one embodiment of the present invention, and Foley catheter;

FIG. 6 is a simplified view showing a typical gravity pipeline with a localized high point as would be known to one of reasonable skill in the relevant art;

FIG. 7 is a simplified view showing a typical bladder driven air-lock conditions as would be known to one of reasonable skill in the relevant art; and

FIG. 8 is a simplified view showing the improved design of the urinary drainage system according to one embodiment of the present invention under typical operating conditions.

The Figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF THE INVENTION

Embodiments of the present invention are hereafter described in detail with reference to the accompanying Figures. Although the invention has been described and illustrated with a certain degree of particularity, it is understood that the present disclosure has been made only by way of example and that those skilled in the art can resort to numerous changes in the combination and arrangement of parts without departing from the spirit and scope of the invention.

The following description with reference to the accompanying drawings is provided to assist in a comprehensive understanding of exemplary embodiments of the present invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. Also, descriptions of well-known functions and constructions are omitted for clarity and conciseness.

Described herein by way of example is an apparatus and associated methodology for an improved urinary drainage system. According to one embodiment of the present invention, a secondary lumen is introduced within the primary tubing connecting the catheter to the collection bag. The secondary lumen is isolated from the primary lumen by a semi-permeable barrier that permits the exchange of the vapor or gas state of a liquid but rejects its liquid phase. As a result, a constant pressure is maintained throughout the length of the tube. This constant pressure enhances fluid flow impeding the onset of bacteria based infections.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Like numbers refer to like elements throughout. In the figures, the sizes of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. Thus, for example, reference to “a component surface” includes reference to one or more of such surfaces.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be also understood that when an element is referred to as being “on,” “attached” to, “connected” to, “coupled” with, “contacting”, “mounted” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

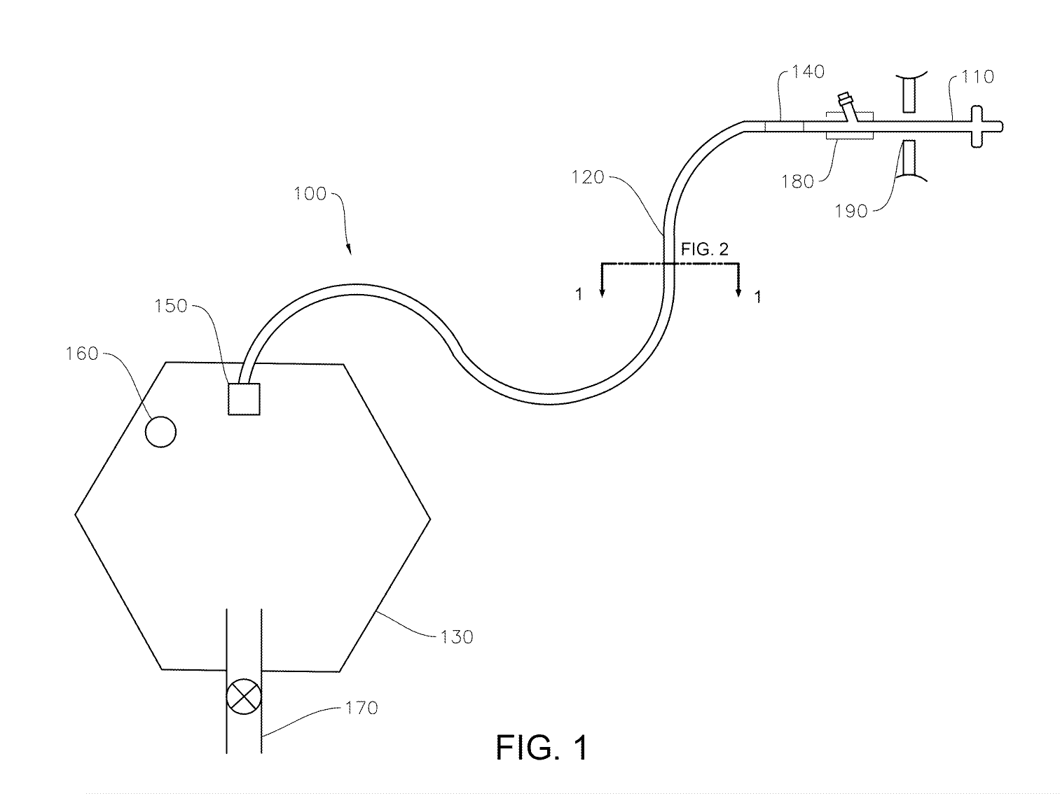

FIG. 1 is a simplified drawing of the urinary drainage system 100, according to one embodiment of the present invention. The system is comprised a Foley catheter (or catheter tip) 110 inserted into a body orifice 190 and secured internally with an inflated balloon and secured externally with a clamped on securement device 180. Fluid is transmitted from the catheter by the drainage system transfer tubing 120, into the collection bag 130. Fluid is routinely drained from the collection bag via the drainage spigot 170. Air enters and exits the collection bag via an encapsulated vent 160, as fluid is drained and fills the bag. The transfer tubing is connected to the Foley catheter via a specifically designed transition fitting 140, and connected to the collection bag at the bag anti-reflux valve fitting 150.

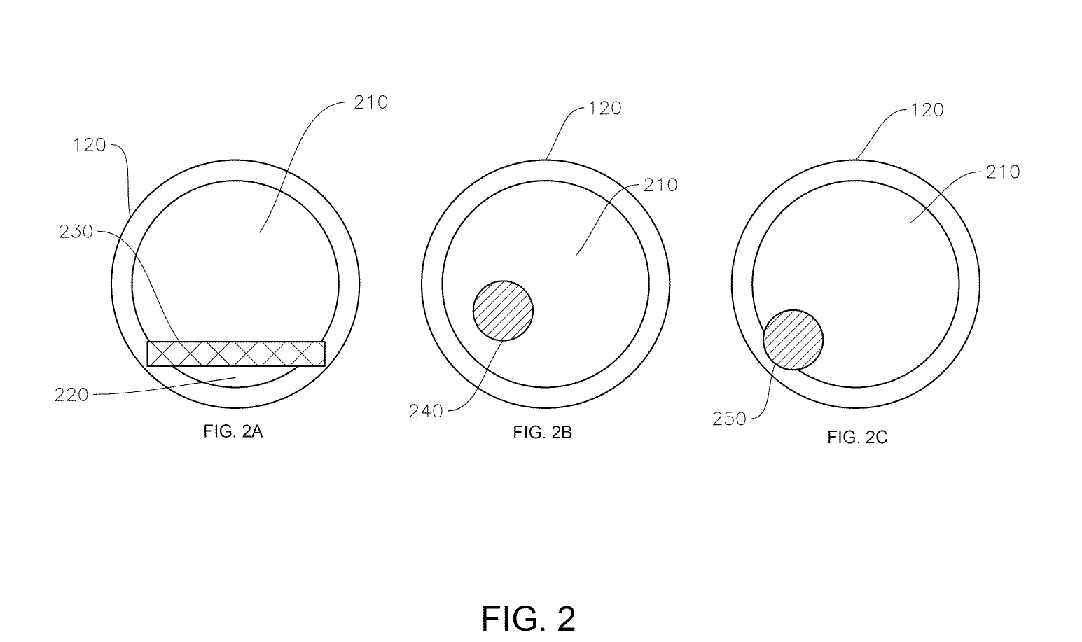

FIG. 2 is a cross-sectional view of the transfer tubing 120 taken along the LINE 1-1 in FIG. 1. In various embodiments, the transfer tubing in the current invention is comprised of a main lumen 210 and secondary lumen 220. In the embodiment depicted in FIG. 2A the secondary lumen 220 is formed with the integral use of a semi-permeable barrier membrane 230. This membrane is comprised of a porous, semi-permeable medical grade material that enables pressure equalization throughout the tube. In the embodiments shown in FIG. 2B and FIG. 2C, the secondary lumen is established with the use of a hollow tube. In FIG. 2B, the secondary lumen 240 is not connected to the transfer tube 120 while in FIG. 2C the secondary lumen 250 is directly connected to interior wall of the transfer tube 120. In all presented embodiments the secondary lumen is present along the entire length of transfer tubing. Alternate cross-sectional shapes of the secondary lumen could be utilized for the same functionality. Similarly, other materials displaying the same physical properties as that described herein for the secondary lumen boundary could be utilized without altering the improved functionality of the current invention.

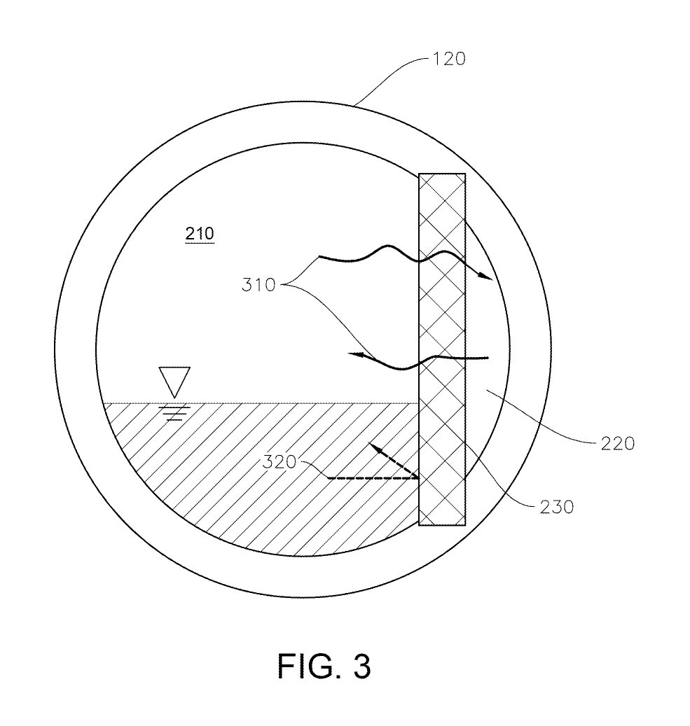

FIG. 3 shows a sectional view depicting the simplified functionality of the secondary lumen according to one embodiment of the present invention. The shown functionality applies to all embodiments of FIG. 2. FIG. 3 displays a typical operational flow regime within the transfer tube 120, whereby two-phased flow exists within the primary lumen 210, comprised of air (vapor) 310, and liquid 320. The secondary lumen 220, includes an interior area that is strictly constituted of air 310. Due to the above described material characteristics of the semi-permeable barrier 230 (and similarly 240 and 250), air 310 freely passes between the primary and secondary lumens, as depicted by the flow arrow showing flow between the two regions. However, the flow of water (liquid), and elemental or ionic constituents is not permitted to pass into the secondary lumen 220. The orientation of secondary lumen (for each embodiment of FIG. 2) will vary depending on the various bends of the flexible transfer tube 120. These orientations will not affect functionality. Similarly, the fraction of air space to liquid space will not affect functionality, but will vary along the length of the transfer tube due to changes in tube slope and fluctuating operational conditions. The nature of the secondary lumen material (either 230, 240 or 250) ensures that when the fluid level changes, the air exposed barrier remains non-wetting and continues to function as an air vent to equalize pressure throught the length of the tube.

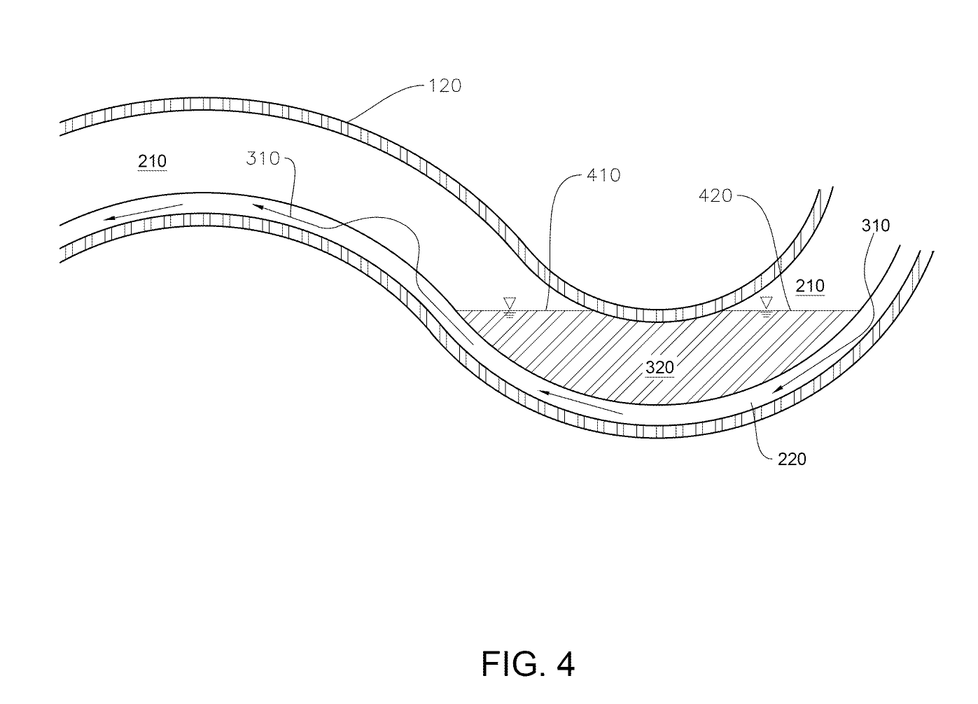

FIG. 4 is a longitudinal view of the transfer tube 120 according to one embodiment of the present invention, and further develops the functionality discussed for FIG. 3. FIG. 4 shows a section of transfer tubing with a first and second air-liquid interfaces 410, 420. As the air upstream of the second interface 420 becomes pressurized (by the action of the bladder, a change in the tubing configuration, or by another means), air above this interface, according to one embodiment of the present invention, moves across the selective membrane 230 and into the secondary lumen 220. The flow arrows in FIG. 4 show this movement of air 310 axially along the transfer tubing within the second lumen 220. Downstream of the first interface 410 the air will move back into the primary lumen 210 and/or proceed axially along the secondary lumen 220, as dictated for the naturally occurring pressure equalization. Whereas, the prior art would balance pressure differences with hydrostatic pressure legs as evidenced by differing fluid levels between the first and second interface 410, 420, the secondary lumen insures balanced pressures and no hydrostatic leg differentials.

The gas permeable nature of the secondary lumen permits air to pass freely from primary lumen to the secondary lumen (and vice versa), so that restrictive, pressurized pockets of air do not form. The porous (semi-permeable) nature of the secondary lumen (as designed) allows for the transfer of air from one section to another to be done in a very subtle, non-disruptive manner. The presence of the secondary lumen does not allow for rushes of air, but rather simple, non-evident adjustments of air volume preventing pressure to build in any section of the tubing. According to one embodiment of the present invention, the hydrophobic/oleophobic (non-wetting) nature of the secondary lumen (as designed) ensures that as the fluid moves from one section of the tube to another, all previous submerged sections of the secondary lumen can function properly as a vent, when no longer entrapped in fluid.

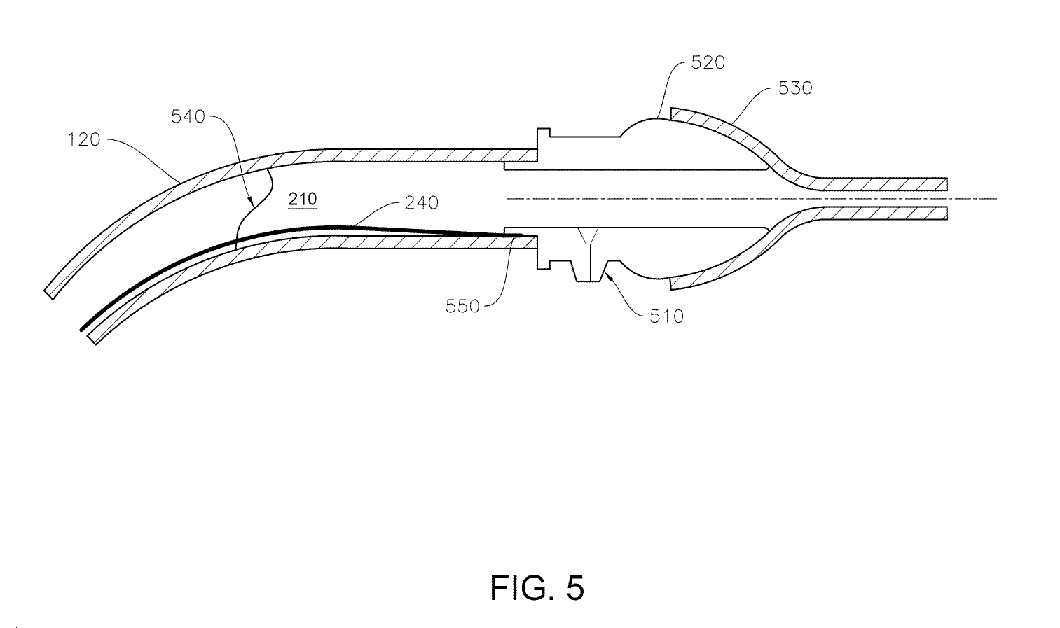

FIG. 5 shows a typical transitional fitting 520 between the transfer tubing 120 and Foley catheter 530. An air-liquid interface 540 occurs distal to the patient. Note specifically, the secondary lumen 240 is intentionally plugged at this transition point 550, ensuring that axial air flow within the secondary lumen 240 occurs only distally (away from) in respect to the patient orifice. The functionality of each possible cross-sectional embodiment from FIG. 2 remains the same with respect to air movement. In this manner, air flow—however subtle—will only occur away from the patient orifice. Any air flow towards the patient might act to transport bacteria towards the bladder—a highly undesirable result. The exact air-liquid interface location will depend on the geometry on the transfer tubing. Typically the interface will set-up just proximal to the first downward bend (as shown) of the transfer tubing. However, due to surface tension effects (see previous discussion on the critical Eotvos number calculations), the interface will not be closer to the patient orifice than the point of the change in diameters, namely, the area of the transitional fitting. The variability of the location of the air-liquid interface (as caused by an upward or horizontal section of transfer tubing) would render a single vent at this location ineffective at removing pressurized air for a large number of clinical scenarios. The feature of continuous venting by the secondary lumen 240 along the entire length of transfer tubing 120 ensures proper pressure equalization regardless of system orientation.

In contrast to the closed end of secondary lumen at the transitinal fitting 550, is the connection of the transfer tubing to the anti-reflux valve, 150. At this location the secondary lumen (220, 240 or 250) remains open to the anti-reflux valve. In this manner, air in the secondary lumen is uninhibited from communicating with the volume of air in the anti-reflux valve and catheter bag.

The standard practice of collecting a urine sample from a sample port 510 located on the transitional fitting, will remain unaltered by the current invention. The standard practice involves doubling the transfer tubing back on itself and temporarily securing the bend with a clamp or hemostat allowing urine to collect between the blockage and the patient. The presence of the secondary lumen in the current invention will not change this practice. The secondary lumen (as designed) in each embodiment will fold and unfold with the transfer tubing, and be restored to its prior shape and function.

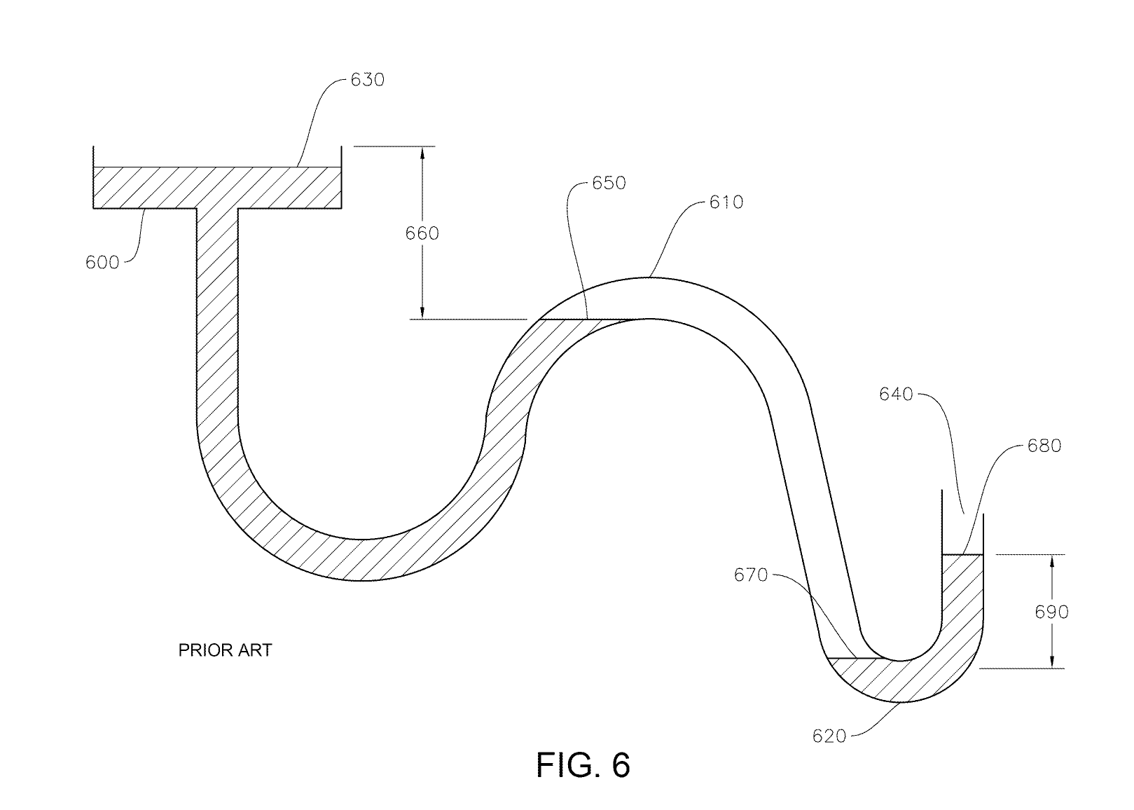

To better understand the technical features and functionality of the present invention, consider the following comparison of prior art configurations of a gravity flow system to that employing the advantages of the present invention. FIG. 6 illustrates a gravity flow pipeline proceeding from an elevated open tank, with an elevated discharge as would be known in the prior art. Patients having standard urinary drainage systems suffer from the effects of air-lock as shown in FIG. 6. Air-locking results from the effects of entrapped air pockets and is accentuated by the surface tension and differential pressure driving delayed drainage as described below.

In the depiction of FIG. 6, an elevated tank 600 is sufficiently large so that the removing of fluid to fill the empty pipeline below, will not alter the height of the surface water elevation 630. Consider for the purpose of this example that the pressure at 630 is atmospheric. As fluid is released into the empty pipeline, it will fill the pipeline and progress towards the high point in the system 610. When sufficient quantity of fluid is released, a first surface 650 at the crest point will be established, and flow will proceed to begin to fill the lowest pipeline point in the pipe 620. When fluid fills the low point 620, a second surface 670 is established, and a fixed mass of air will be trapped within the pipeline between 650 and 670. As more fluid is released, the second surface 670 will naturally rise towards the direction of the high point 610. The fixed mass of entrapped air will be forced to decrease in volume, and thereby increase in pressure. As shown in FIG. 6, the surface heights at the second surface an 670 and a corresponding third surface 680, will differ in elevation to accommodate the differential pressure. In this example the end of the pipeline 640 is open to atmospheric pressure. The consequence is that fluid about the low point 620 will function like a simple manometer, with the difference of surface elevations 690, becoming a direct reflection of the differential pressure of the entrapped mass of air between the first surface 650 and the second surface 670 combined with surface tension effects.

In essence, FIG. 6 represents a real-world, tangible example of the potential for an air-locked system. Initially it may be counter-intuitive to contemplate that a tank full of water upon a hillside would not by gravity alone drain through a pipeline to an area below. However, in the situation where the pipeline is originally empty of fluid, a special case exists. With a high point 610 in the pipeline, a pocket of air will become trapped as low velocity fluid trickles downhill from the tank. This air pocket 610,670 can act to block the flow of fluid as it is forced to decrease in size as the pipeline fills. In the actual practice of the design of such gravity pipelines, a vent would be placed at highpoint 610. As the air space begins to pressurize during filling of the line, this vent would exhaust the air (only) until the line was filled with fluid, in which case, flow would proceed as is intuitive, from high to low places. Multiple high points within such a system would require multiple vents. With the prior art urinary drainage system a simple vent is not possible, however, because the highpoints of the system are ever changing with the movement of the tubing. Furthermore, placing additional holes (vents) within such a system would provide additional avenues for bacterial contamination from the outside environment, a highly undesirable condition.

FIG. 6 is drawn showing the critical case condition for potential air-lock. The pipeline length at the exit point 640 is sufficiently high that water surface elevation 690 does not rise to flow out. Similarly, the pressure generated by elevated tank 600 and high point 610, is not sufficient to lower the fluid surface elevation as the second surface 670 to where the entrapped air can escape at the low point 620. In this critical case, the height of the elevated tank 600 is not enough to continue fluid flow and the system flow becomes static as an air-locked state exists.

Utilizing the engineering principal that hydrostatic pressure is the product of fluid specific weight and the depth of the fluid column, the air-locked condition will occur in an atmospheric system as shown in FIG. 6, when the pressure generated by column 690 exceeds the pressure created by the tank height 660. Since the fluid specific weights are identical, air-lock occurs when height 690 exceeds height 660. In essence, air-lock occurs when the downstream pressure resistance overcomes that provided by the tank. Note that if the system had multiple high points, as in an undulating channel, the driving pressure provided by the tank and first high point would be further reduced by the height of each successive high point. The point worth noting is that with a complicated, undulating terrain the propensity for air-lock is increased.

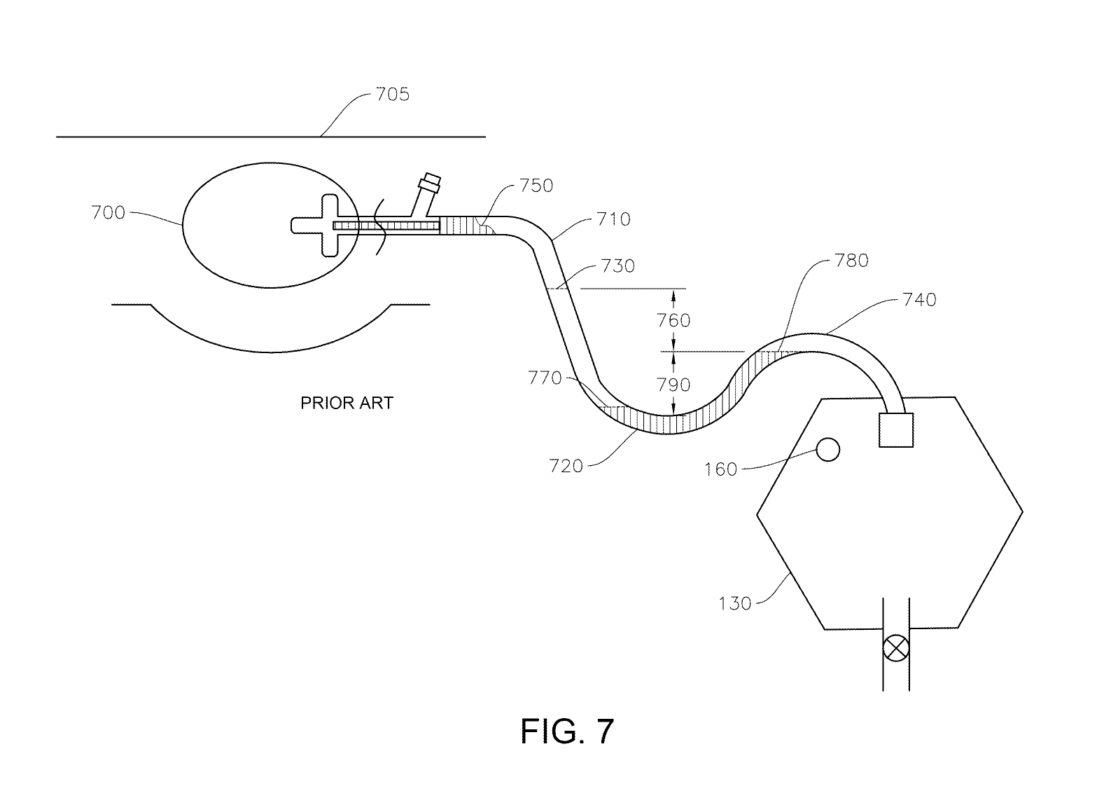

The analogy presented in FIG. 6 is translated in the actual clinical conditions shown for patient 705 with an inserted Foley catheter, in FIG. 7. The fact that the bladder 700 itself, can generate a system pressure as it contracts in the forcing of urine outward, eliminates the need for a specific high point. As discussed previously for FIG. 5, the physical properties of the catheter and transfer tubing will result in a urine-air interface 750 prior to the point of the first downward tubing bend 710. This physical characteristic ensures the possibility of a trapped mass of air between surface 750 and 770. As fluid fills the system low point 720, the pressure provided by the bladder is expressed as the height difference between surface 780 and 770. Note that as drawn, FIG. 7 represents the critical condition with respect to surface 770 and 780. Pressure in the collection bag 130 and endpoint tubing 740, is atmospheric due to the function of the bag vent 160. A direct measure of the applied bladder pressure is thus height 790, which is the simple difference in elevation from surface 780 and 770. Clinical observations demonstrate that with a catheter in place, the normal human bladder can provide no more than 6 inches water column (WC) pressure (which is 0.217 psig, in a more common pressure unit). Thus, an air-locked condition will occur if height 790 is greater than 6 inches of height. The depiction in FIG. 7 represents the simplest tubing configuration of a single dependent loop. As described previously, multiple intermediate high and low point serve to make the system air-lock more easier.

FIG. 7 presents an application of the scenario shown in FIG. 6 to a gravity flow urinary collection device. FIG. 7 includes an intermediary surface 730 as an alternate condition to the second surface 770. In this alternate configuration urine will exist between the intermediary surface 730 and a third surface 780 proximal to the collection bag 130. This condition is observed clinically, and is the result of the catheter tip becoming plugged by bladder tissue or other means. With a change of tubing location or other system condition modification, the trapped mass of air between the intermediary surface 730 and the distal end of the catheter 750 will decrease in pressure below atmospheric. The amount of the pressure drop is reflected in the difference 760 in heights between the intermediary surface 730 and the third surface 780. In the typical clinically observed cases, the fluid level at the third surface 780 will not be located at the crest point of the tubing into the collection bag, but rather positioned somewhat nearer to a low point 720. The sub-atmospheric pressure in the entrapped air and catheter tip perpetuates the air-lock by keeping the obstructing tissue sucked against the catheter holes.

The transfer tubing utilized in all typical drainage systems is necessarily small in diameter to make surface tension effects of sufficient magnitude to compete with gravitational effects. These effects result in the creation of pressurized pockets within the tube impeding the fluid flow as a result of the interaction of surface tension effects. The arrested flow that results from the surface tension effects in the transfer tubing creates pressured air pockets, or expressed another way, stationary air bubbles. Recall that the cohesive forces among liquid molecules are responsible for the phenomenon of surface tension. In the bulk of the liquid, each molecule is pulled equally in every direction by neighboring liquid molecules, resulting in a net force of zero. The molecules at the surface do not have other molecules on all sides of them and therefore are pulled inwards. This creates some internal pressure and forces liquid surfaces to contract to the minimal area. Surface tension is responsible for the shape of liquid droplets.

Capillary action (sometimes capillarity, capillary motion, or wicking) is the ability of a liquid to flow in narrow spaces without the assistance of, and in opposition to, external forces like gravity. The effect can be seen in the drawing up of liquids between the hairs of a paint-brush, in a thin tube, in porous materials such as paper, in some non-porous materials such as liquefied carbon fiber, or in a cell. It occurs because of intermolecular forces between the liquid and surrounding solid surfaces. If the diameter of the tube is sufficiently small, then the combination of surface tension (which is caused by cohesion within the liquid) and adhesive forces between the liquid and container act to lift the liquid. In short, the capillary action is due to the pressure of cohesion and adhesion that cause the liquid to work against gravity. For example, when the lower end of a vertical glass tube is placed in a liquid, such as water, a concave meniscus forms. Adhesion occurs between the fluid and the solid inner wall pulling the liquid column up until there is a sufficient mass of liquid for gravitational forces to overcome these intermolecular forces. The contact length (around the edge) between the top of the liquid column and the tube is proportional to the diameter of the tube, while the weight of the liquid column is proportional to the square of the tube's diameter. So, a narrow tube will draw a liquid column higher than a wider tube.

Turning back to example illustrated in FIG. 7, as the supine patient in bed moves, or as care providers alter the position of the tubing, the size and location of these air pockets will correspondingly change. Using the previous analogy, it is as if the pipeline terrain is continually changing. Depending on the position of the tubing, the compressible air in these pockets will enlarge or contract (as will the pressure inside the bubble) to accommodate the incompressible urine surrounding them. In effect, this continually altering terrain causes a stationary pressure differental in the urinary drainage tubing that impedes the free flow of a fluid. In addition the small diameter nature of the transfer tubing results in surface tension effects on the same order of magnitude as gravity forces creating a suspended column of fluid. This suspended column of fluid results in further retardation of ante-grade flow.

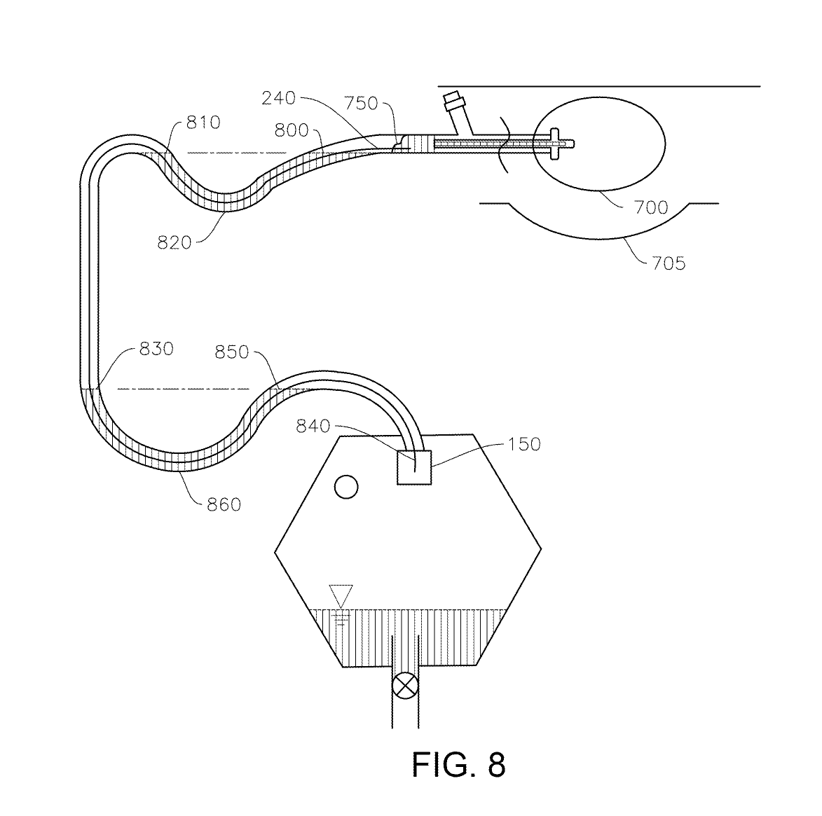

As this discussion shows, air-lock in the urinary drainage system is a troublesome problem with the systems of the prior art. It is not simply the existence of dependently looped tubing that causes the air-lock problem, but also the complex existence of tubing undulations, pressure fluctuations in air pockets, the retarding surface tension effects, and bladder pressurization. FIG. 8 shows a typical complex configuration of tubing utilizing the pressure dissipating feature according to one embodiment of the present invention. As shown in FIG. 8, pressurized air pockets are resolved through the presence of an inter-luminal semi-permeable barrier, which ensures continuous ante-grade flow free of air-locking Flow that naturally fills intermediate low spots 820, 860 will have surface combinations 800/810, 830/850 that are balanced in height. On each side of these surfaces there will be equal pressure, as dictated by the presence of the secondary lumen 240. In the embodiment shown in FIG. 8 the secondary lumen distal end 840 terminates into the anti-reflux valve 150 creating an atmospheric port for the secondary lumen 240. The present invention, as presented herein, eliminates the air-lock potential by eliminating the pressurized air sections within the transfer tubing.

The presence of tubing undulations does not affect functionality since pressure build-up is eliminated and the flow is driven simply by gravity in an ante-grade fashion. As the body delivers more fluid into the bladder, the bladder compresses as is normally done without a catheter in place. Flow simply cascades from section to section in the transfer tubing, and finally into the collection bag. The small total volume of transfer tubing ensures that minimal urine is held between the bladder and the collection bag.

The modified drainage system, as proposed by the present invention, minimizes the potential for retrograde bacterial introduction into the bladder (by aerosol, liquid flow or yet unidentified means) by eliminating pressure build-up from all sections of the transfer tubing. The porous, gas permeable nature of the secondary lumen provides a built-in “buffering” which will eliminate pressured “rushes” of air about the transfer tubing, as the patient moves or the bag or tubing are moved. This will minimize pressurized aerosol fluctuations as a potential vector for bacterial transmissions. Furthermore, the inherent resistance to two-phase flow within the small diameter of catheter tubing, which is result of the sub-critical Eotvos number (see TABLE 2 and discussion therein), will also be maintained since the potential for an elevated pressure between fluid surfaces 750, 800 has been eliminated.

Note that the improved system maintains the historically proven critical feature of a “closed system.” The invented system does not introduce any additional external openings to the outside environment which would allow external contamination. The secondary lumen acts as a venting system which is completely internal to the system boundary, and thus the title “closed system internal vent” adequately highlights the improved performance.

In clinical practice the elimination of dependent loops in the transfer tubing will benefit efficient flow towards the drainage bag. This benefit applies to the improved system as well as the current system. However, with the current invention the debilitating effects of the dependent loops have been eradicated. In essence the “power” of the dependent loops to significantly retard flow and transmit aerosolized bacteria has been eliminated.

In order to effectively prove the performance of proposed innovation, a bench top test model was developed for controlled observations. The human bladder was modeled by modifying a plastic 1 Liter IV fluid bag by boring open a larger hole in the standard spike port. Through this opening a 16 French catheter was threaded, and its balloon inflated. The penetration point was sealed with silicone caulk to create a water proof connection. This intravenous bag was then filled with fluid and all air removed. A simple pinch clamp was place across the catheter diameter to allow for the controlled initiation of flow at the start of the test.

Bench tests were conducted using the prior art transfer tubing and bag, and prototype models of the present invention. In each parallel comparison, identical tubing curvatures and component locations were established for a true comparison. For these trials the embodiment depicted by FIG. 2C was utilized with the secondary lumen simply extended into the anti-reflux valve. Comparative tests were done in a parallel fashion using separate bladder models, and then confirmed in a sequential timed test using the same bladder on each system. The urinary drainage system was positioned such as to have the Foley catheter and bladder model resting horizontally on a table, with the collection bag hung at a lower level. The transfer tubing proceeded horizontally from the table down into a single dependent loop. The transfer tubing then arose from the bottom of the dependent loop, cresting into the collection bag. This arrangement represents the typical clinical use of the urinary drainage system of a patient lying in bed and the collection bag hanging from provided hooks on the patient's bedframe (see FIG. 7). It was determined that a 2.5 pound weight positioned on a horizontally resting bag bladder model generated about 6.5 inches WC pressure, which is in the range of observed normal bladder function.

The first parallel test was conducted with two system in the exact same physical configure. The transfer tubing of both systems was configured with a dependent loop height of 8 inches. See dimension 790 in FIG. 7, as a pictorial representation. With each system secured in place, and the pressurizing weight atop the bladder model, the pinch clamps for each system were simultaneously released allowing flow. Liquid flow through the prior art system filled the bottom of the dependent loop and set up a pressure differential consistent with the 6.5 inches WC pressure applied. The system quickly became air-locked as no flow was able to crest the top of the dependent loop into the collection bag. This system quickly became static. In contrast, flow proceeded through the invented system and continued until the applied weight bottomed out against the supporting table. The fluid progressed from the bladder filling the collecting bag. In both cases, a clear liquid-air transition existed distal to catheter-transfer tube junction (see FIG. 5 depiction), confirming that an artificial siphon had not been developed. A subsequent test was conducting using the same bladder model on each system independently, which confirmed the results.

A similar parallel test was conducted with an alternative configuration. This test was conducted in a manner similar to that described above, except that the transfer tubing of both systems was positioned with a dependent loop measuring 4 inches. This height was less than the critical maximum of 6.5 inches for the chosen applied weight. When the pinch clamps were simultaneously released from the liquid filled bladders, both system flows proceeded to crest the tubing and fill the collection bags, as expected. Both systems ran until the applied weight bottomed out. The current invention drained faster than the prior art as measured by bag volume in approximately a 2:1 ratio throughout the test. This test showed that the current invention reduces the retarding effects of the dependent loop. As before, results were confirmed by testing each system independently with the same bladder.

The scenario presented as an alternate condition in discussion about FIG. 7 (plugged catheter tip openings) was also replicated on the bench top. With each bladder full of liquid and depleted of air, the pinch clamps were opened allowing approximately 25 ml of fluid to proceed and fill the bottom of the primary dependent loop. The pinch clamps were then closed. This situation models the condition of when the catheter tip is plugged within the bladder. Slowly lowering the collection bag and distal tubing, resulted in pressure legs of differing height in the prior art system, identical to that presented in FIG. 7 for the two fluid surfaces 730, 780. This result was as expected for the prior art, as the mass of trapped air expanded in volume resulting in a below atmospheric pressure drop which secures the catheter tip seal against the bladder mucosa. The improved design urinary drainage system however responded differently. With the movement of the bag and tubing, the pressure legs quickly adjusted and remained at the same height. As expected, the current invented system did not allow a pressure difference to form. In actual patient usage, the improved system characteristic will allow the catheter tip to more easily disengage from an obstructing bladder wall surface, and resume normal ante-grade flow. The result is improved flow and no bladder wall damage.

To examine whether the present invention enhanced retrograde flow, a test was conducted by measuring the time a small volume of liquid takes to travel through the transfer tubing from an elevated location down to the transitional fitting and Foley catheter (in parallel systems of current invention and prior art). This testing was done to mimic what might occur (in the clinical setting), if the collection bag was errantly elevated above the patient's bladder. Results showed no significant decrease in this flow time of the current invention as compared to the prior art. This is due to the fact that the small diameter of the catheter and dead-end secondary lumen provide resistance which dominates the flow characteristics. In order for the proposed transfer tubing to drain fluid more effectively there needs to be a reservoir or cavity of air to receive and dissipate the displaced air. The anti-reflux and collection bag provide this cavity for ante-grade flow. However with an elevated collection bag (forbidden in actual practice), no such cavity exists. Rather, as previously described, the secondary lumen is intentionally sealed closed at the transition fitting. The air-liquid transition that naturally sets up directly outside the bladder (see FIG. 5), results in no significant air volume cavity. The result is that retrograde towards the bladder is essentially the same between the prior art and proposed system. This substantiates the benefit of the innovated system.

Although the invention has been described and illustrated with a certain degree of particularity, it is understood that the present disclosure has been made only by way of example and that those skilled in the art can resort to numerous changes in the combination and arrangement of parts without departing from the spirit and scope of the invention.

It is also is recognized that the teachings of the foregoing disclosure will suggest other modifications to those persons skilled in the relevant art. Such modifications may involve other features that are already known per se and which may be used instead of or in addition to features already described herein. Although claims have been formulated in this application to particular combinations of features, it should be understood that the scope of the disclosure herein also includes any novel feature or any novel combination of features disclosed either explicitly or implicitly or any generalization or modification thereof which would be apparent to persons skilled in the relevant art, whether or not such relates to the same invention as presently claimed in any claim and whether or not it mitigates any or all of the same technical problems as confronted by the present invention. The Applicant hereby reserves the right to formulate new claims to such features and/or combinations of such features during the prosecution of the present application or of any further application derived there from.

I claim:

1. A system for enhanced drainage of bodily fluids, the system comprising:
a fluid extraction device operable to remove a fluid from a fluid source;
a fluid collection device; and
a length of a conduit fluidly coupling the fluid extraction device to the fluid collection device, wherein the conduit includes an interior channel and wherein the interior channel is separated along the length of the conduit by a selectively permeable membrane defining a primary lumen and a secondary lumen.

2. The system for enhanced drainage of bodily fluids according to claim 1, wherein the fluid extraction device is a catheter.

3. The system for enhanced drainage of bodily fluids according to claim 1, wherein the selectively permeable membrane is permeable to a vapor state of a substance and impermeable to a liquid state of the substance.

4. The system for enhanced drainage of bodily fluids according to claim 3, wherein the secondary lumen is void of water.

5. The system for enhanced drainage of bodily fluids according to claim 1, wherein the selectively permeable membrane maintains throughout the length of the conduit equal pressure.

6. The system for enhanced drainage of bodily fluids according to claim 1, wherein the fluid is urine.

7. The system for enhanced drainage of bodily fluids according to claim 6, wherein the selectively permeable membrane is permeable to air and impermeable to urine.

8. The system for enhanced drainage of bodily fluids according to claim 7, wherein the secondary lumen if void of urine.

9. The system for enhanced drainage of bodily fluids according to claim 1, wherein the secondary lumen is operable to equalize pressure throughout the length of the conduit.

10. The system for enhanced drainage of bodily fluids according to claim 9, wherein the secondary lumen is vented to the fluid collection device.

11. The system for enhanced drainage of bodily fluids according to claim 1, wherein the selectively permeable membrane reduces aerosol transmission of bacteria within the conduit.

12. The system for enhanced drainage of bodily fluids according to claim 1, wherein the selectively permeable membrane eliminates vacuum driven adherence of a catheter tip to a bladder mucosa.

13. The system for enhanced drainage of bodily fluids according to claim 12, wherein the selectively permeable membrane eliminates urine trapped within a bladder.

14. A system for pressure equalization in a delivery tube, the system comprising:
a source of a fluid;
a receptacle for collection of the fluid; and
a conduit fluidly connecting the source of the fluid to the receptacle wherein the conduit includes a first lumen and a second lumen and wherein at least a portion of the first lumen is separated from the second lumen by a selectively permeable membrane.

15. The system for pressure equalization in a delivery tube according to claim 14, wherein the source of the fluid is a catheter.

16. The system for pressure equalization in a delivery tube according to claim 15, wherein the catheter is a Foley catheter.

17. The system for pressure equalization in a delivery tube according to claim 14, wherein the fluid is urine.

18. The system for pressure equalization in a delivery tube according to claim 14, wherein the selectively permeable membrane is permeable to a vapor state of a substance and impermeable to a liquid state of the substance.

19. The system for pressure equalization in a delivery tube according to claim 14 wherein the conduit maintains constant pressure.

20. The system for pressure equalization in a delivery tube according to claim 19, wherein the secondary lumen equalizes pressure throughout the conduit.

21. The system for pressure equalization in a delivery tube according to claim 14, wherein the secondary lumen terminates within the receptacle.
